(19) United States
(12) Patent Application Publication
(10) Pub. No.: US 2022/0054111 A1
(43) Pub. Date: Feb. 24, 2022

(54) SYSTEMS AND METHODS FOR PERFORMING TISSUE BIOPSY

(71) Applicant: THE REGENTS OF THE UNIVERSITY OF MICHIGAN, Ann Arbor, MI (US)

(72) Inventors: Dian-Ru Li, Ann Arbor, MI (US); Jeffrey Stephen Plott, Algonac, MI (US); Jeffrey Montgomery, Ann Arbor, MI (US); Albert J. Shih, Ann Arbor, MI (US)

(21) Appl. No.: 17/312,501

(22) PCT Filed: Dec. 10, 2019
(86) PCT No.: PCT/US2019/065359

§ 371 Date: Jun. 10, 2021

Related U.S. Application Data
(60) Provisional application No. 62/778,066, filed on Dec. 11, 2018.

(51) Int. Cl. A61B 10/02

(52) U.S. Cl. CPC A61B 10/0241

(57) ABSTRACT

The present disclosure relates to devices, systems, and methods for performing needle biopsies. In particular, provided herein is a biopsy device comprising an asymmetric stylet tip with multiple bevels and uses thereof.

### CROSS REFERENCE TO RELATED APPLICATIONS

This application claims the benefit of U.S. Provisional Application No. 62/778,066, filed Dec. 11, 2018, which is incorporated herein by reference in its entirety.

### STATEMENT REGARDING FEDERALLY SPONSORED RESEARCH OR DEVELOPMENT

This invention was made with government support under CMMI1266063 awarded by the National Science Foundation. The government has certain rights in the invention.

### FIELD OF THE DISCLOSURE

The present disclosure relates to devices, systems, and methods for performing needle biopsies. In particular, provided herein is a biopsy device comprising an asymmetric stylet tip with multiple bevels and uses thereof.

### BACKGROUND

Needle biopsy is a common medical procedure to obtain tissue samples from the targeted organ, such as the liver, lung, breast and prostate, for cancer diagnosis. Accurate needle deployment and adequate tissue sampling in biopsy are essential for accurate diagnosis and individualized treatment decisions. Advances in medical imaging, particularly magnetic resonance imagining (MRI), have enabled early identification of suspicious cancerous lesions which then require targeted needle biopsy to sample the identified lesion site for subsequent confirmatory pathological diagnosis. The tissue sampling accuracy and adequacy depends on the needle deployment at the targeted sampling site and needle-tissue interaction during the tissue cutting/sampling process, respectively.

Biopsy procedures are generally performed using a hand-held trucut needle device with two major coaxial components: a solid stylet (inside) and a hollow needle (outside). The stylet commonly has a single bevel tip and a groove on the same side of that bevel which stores the tissue sample. The mechanical springs in the device trigger the stylet and needle sequentially at a high speed to cut the tissue and trap it inside the groove.

However, achieving the desired millimeter (mm) and sub-mm needle deployment accuracy is still clinically and technically challenging. The existing single-bevel stylet tip can yield an adequate tissue sample amount but often leads to stylet deflection due to the unbalanced forces and bending moments during insertion into and through tissue. The outer needle follows the deflected stylet to sample the tissue, causing variance between the targeted and actual locations of the resultant tissue core and contributing to lesion undersampling/missampling. Such sampling errors can lead to false negative biopsy results, misdiagnosis and delay in treatment, negatively impacting the patient's quality of life.

Biopsy devices with improved needle deployment accuracy while maintaining adequate tissue sampling are needed.

### SUMMARY OF THE DISCLOSURE

The present disclosure relates to devices, systems, and methods for performing needle biopsies. In particular, provided herein is a needle biopsy device comprising an asymmetric multi-bevel stylet tip and uses thereof.

The needle biopsy devices described here address the deficiencies of existing devices in deployment accuracy. By using an asymmetric stylet tip with a plurality of bevels, the devices described herein reduce deflections while maintaining an adequate tissue sampling amount compared to that of the existing single-bevel stylet currently in widespread use.

For example, in some embodiments, provided herein is a biopsy device, comprising: a stylet comprising a stylet tip, wherein the stylet tip comprises at least two bevels and an initial cutting element, wherein at least two of the bevels are radially asymmetric, and wherein the bevels converge to form the initial cutting element. In some embodiments, the stylet further comprises a tissue storage groove comprising a tissue groove face, wherein the tissue storage groove is configured for storage of tissue obtained during a biopsy.

The present disclosure is not limited to any particular initial cutting element. Examples include, but are not limited to, a single cutting point, a horizontal cutting edge, or a vertical cutting edge. In some embodiments, the initial cutting element is below or at least partially aligned with the tissue groove face. In some embodiments, the at least two bevels comprise at least one primary bevel and at least one balancing bevel(s). In some embodiments, a primary bevel is a bevel with a normal surface component at least partially in the same direction as a normal surface component created by the groove face. In some embodiments, a balancing bevel is a bevel with at least a portion of a normal surface component that is not at least partially in the same direction as a normal surface component created by the groove face. In some embodiments, the primary bevel is on the same side of the tissue storage groove. In some embodiments, a bevel is curved such that it acts as both a primary and balancing bevel. In some embodiments, the one or more balancing bevels generate a force which at least partially opposes the forces generated by the one or more primary bevels and the non-uniform tissue compression during tissue penetration. In some embodiments, the primary and balancing bevel(s) form a continuous surface on the tip.

The present disclosure is not limited to particular bevel shape or arrangement. Examples include but are not limited to: one primary bevel and three balancing bevels, one primary bevel and one balancing bevel, one primary bevel and two balancing bevels, two primary bevels and one balancing bevel. In some embodiments, the bevels comprise a bevel shape (e.g. curved face or flat surface), angle and bevel length. In some embodiments, the balancing bevels comprise the same or different bevel shape, angle and bevel length. In some embodiments, the balancing bevels comprise the same or different bevel shape, angle and bevel length as the primary bevel. In some embodiments, the balancing bevels are oriented at plus or minus 90-180° around the center line of stylet cylindrical body relative to the primary bevel. In some embodiments, the bevels comprise a bevel angle of 10-25°. In some embodiments, the total surface area of the balancing bevels is larger than 20% of the area of the primary bevels (e.g., to provide sufficient forces to balance the bending instability), in contrast to a needle lancet point wherein additional small bevels are introduced to a stylet or needle tip to increase the tip sharpness. In some embodiments, the initial cutting element is below the groove face by 10-70% of the groove thickness. In some embodiments, the biopsy device further comprises a hollow needle and a deployment component (e.g., in the device body), which advances the stylet and needle sequentially at a high speed to cut the tissue and trap it inside the tissue groove during biopsy procedure, wherein the deployment component is, for example, a spring, pneumatic source, hydraulic source, or motor. In some embodiments, the biopsy device exhibits decreased deflection during deployment relative to a biopsy device lacking radial asymmetric balancing bevels (e.g., in some embodiments, the biopsy device exhibits less than 1 mm (e.g., less than 0.52 or 0.5 mm deflection of a 1 mm stylet). In some embodiments, deflection is reduced by at least 50% (e.g., at least 55%, 60%, 65%, 70% or more) when compared to a biopsy device lacking the radial asymmetric balancing bevels. In some embodiments, the biopsy deice exhibits similar tissue sampling amounts when compared to a biopsy device with a single bevel tip.

In further embodiments, provide herein is a stylet comprising a stylet tip, wherein the stylet tip comprises at least two bevels and an initial cutting element, wherein at least two of the bevels are radially asymmetric, and wherein the bevels converge to form the initial cutting element.

Also provided herein is a system, comprising a stylet tip and a stylet, wherein the stylet tip is attached to or configured to be attached to, a stylet.

Further embodiments comprise methods and uses of obtaining a tissue biopsy sample, comprising: deploying a biopsy device as described herein to obtain a tissue sample (e.g., from liver, kidney, breast, lung, or prostate tissue). In some embodiments, the tissue is cancerous or suspected of being cancerous.

Additional embodiments are described herein.

### BRIEF DESCRIPTION OF DRAWINGS

FIG. 1 shows an exemplary targeted biopsy procedure of co-registered MRI-ultrasound fusion prostate needle biopsy wherein the suspicious cancer lesion (diseased tissue) has been identified by the pre-biopsy MRI, which is then fused into the live ultrasound image to provide real-time needle guidance, wherein the stylet insertion forces cause significant stylet deflection during the high-speed insertion (firing) deviating from the ideal insertion path, wherein the outer needle follows the deflected stylet to undersample/missample the targeted cancerous lesion.

FIG. 2A, FIG. 2B, FIG. 2C and FIG. 2D show the steps of a needle biopsy procedure using a stylet with a single bevel tip and the insertion forces causing stylet deflection, which can contribute to the missampling of the cancerous tissue site.

FIG. 3A, FIG. 3B, FIG. 3C and FIG. 3D show the steps of a needle biopsy procedure using a stylet with asymmetric multi-bevel tip (present disclosure) and the multi-directional insertion forces to balance the bending forces and moments. Because of this balance, a straighter sampling path is achieved which allows the needle to accurately sample the targeted cancerous site.

FIG. 4A-FIG. 4B shows an example of the deployment path with deflection of FIG. 4A) a single beveled stylet (currently in clinical use) and FIG. 4B) an asymmetric multi-beveled stylet tip (the present disclosure).

FIG. 5 shows a stylet of an exemplary embodiment of the present disclosure wherein the stylet comprises of a primary bevel and three balancing bevels.

FIG. 6 shows a close-up view of the stylet tip of FIG. 5.

FIG. 7 shows a diagram of bevel radial orientation and bevel angle for each bevel on an asymmetric multi-beveled stylet tip.

FIG. 8 shows a front view of an exemplary stylet tip with one primary bevel and three balancing bevels.

FIG. 9 shows top, side, and bottom views of an exemplary stylet tip with one primary bevel and three balancing bevels.

FIG. 10 shows a detailed side view of the stylet of FIG. 9

FIG. 11 shows three different exemplary asymmetric stylet tip designs with different initial cutting elements (top: a single point; middle: horizontal cutting edge; bottom: vertically tilt cutting edge).

FIG. 12A shows an exemplary stylet tip with one primary bevel and one balancing bevel. Shown is a side view (top), and isometric views (middle and bottom). FIG. 12B and FIG. 12C show additional stylet tip designs wherein the stylet comprises a primary bevel and two balancing bevels.

FIG. 13 shows an isometric view of an exemplary stylet tip with two primary bevels and one balancing bevel.

FIG. 14 shows isometric, side, and front views of an exemplary stylet tip with the concave bevel shape.

FIG. 15 shows isometric and side views of an exemplary stylet tip with the convex bevel shape.

FIG. 16 shows isometric and side views of an exemplary stylet tip with continuous primary and balancing bevels.

FIG. 17A and FIG. 17B show a needle biopsy device of an exemplary embodiment of the present disclosure wherein the device further comprises a hollow needle and a spring-loaded mechanism in the device body, and FIG. 17C shows the device advancing the stylet and needle sequentially during biopsy procedure.

FIG. 18 shows a comparison of stylet deflection of the stylet of FIG. 8 and a single bevel stylet tip (currently in clinical use) in the healthy tissue mimicking material.

FIG. 19 shows a comparison of deployment and deformation of the stylet of FIG. 8 and a single bevel stylet tip (currently in clinical use) after inserting into the cancerous tissue mimicking material.

FIG. 20A-FIG. 20E shows four different stylet tip geometries and their corresponding tip face forces and optical microscopy images, wherein FIG. 20A shows a single-bevel (SB) stylet, and FIG. 20B, FIG. 20C, and FIG. 20D show three asymmetric multi-bevel stylets, low multi-beveled (LMB), aligned multi-bevel (AMB), and high multi-bevel (HMB) stylets, respectively, wherein LMB, AMB, and HMB stylets have the initial cutting element lower than (present disclosure), aligned with, and higher than the groove face, respectively. FIG. 20E shows the schematic diagram to define the stylet parameters of t, d_{b}, dₜ, l_{g}, and t_{g}.

FIG. 21 shows the mean values of stylet deflection δ (top) and tissue sampling length lₛ and weight wₛ (bottom) in three tissue-mimicking phantoms, each with a different hardness, for the SB, LMB, AMB, and HMB needles (error bars represent the corresponding standard deviations) and the images in needle deflection and stylet deflection experiments.

FIG. 22 shows the mean values of tissue sampling length in cadaver prostate tests for SB and LMB needles.

### DETAILED DESCRIPTION

The present disclosure relates to devices, systems, and methods for performing biopsies. In particular, provided herein is a biopsy device comprising an asymmetric stylet tip and uses thereof.

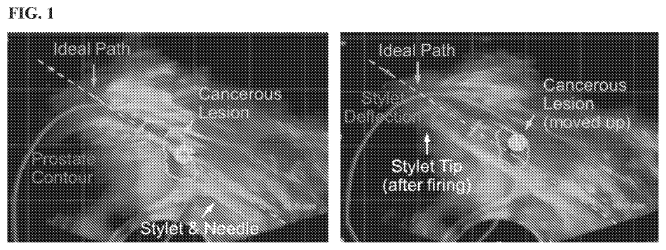

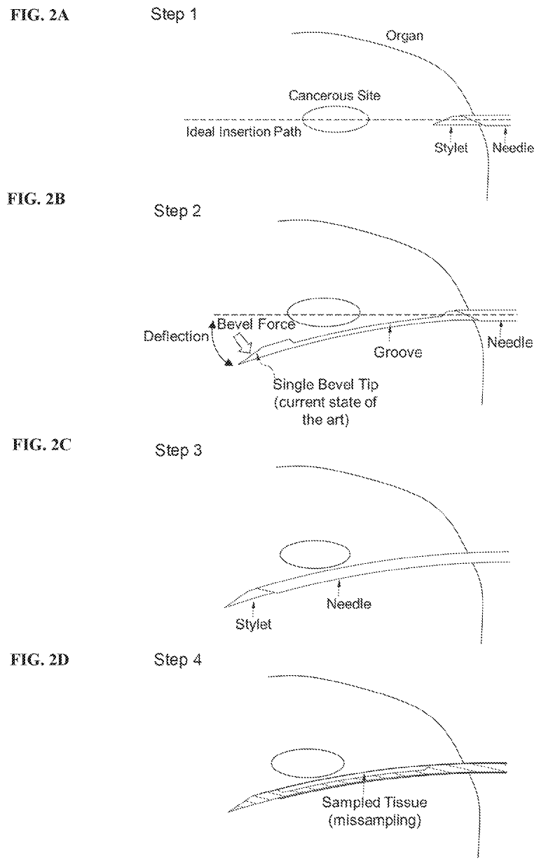

Needle biopsy is commonly performed with a trucut needle biopsy device, also called an automatic, spring-loaded biopsy instrument, which includes an inner solid stylet connected to a trough, or shallow receptacle, covered by an outer hollow needle and attached to a spring-loaded mechanism. As shown in FIG. 1, in an exemplary biopsy procedure such as, for example, co-registered MRI-ultrasound fusion targeted prostate biopsy, a suspicious cancer lesion site has been identified by a pre-biopsy MRI. The pre-biopsy MRI is then fused into a live ultrasound image to provide real-time needle guidance with the ideal biopsy path overlaid on the display for the clinician to follow to sample the targeted and/or cancerous lesion (lesion center is displayed as a target for aiming). However, existing stylets with single bevel tip geometry bend or deflect during and after deployment, deviating away from the ideal straight path. The cancerous lesion is also moved due to this deflection. The outer needle then follows the deflected stylet, undersampling/missampling the targeted sampling site. Such tissue sampling errors can potentially lead to false negative results (e.g., the targeted and/or cancerous lesion site is not accurately sampled) and cancer misdiagnosis. FIG. 2 shows the steps of a needle biopsy procedure using a stylet with a single bevel tip as is commonly used in current practice. First (FIG. 2A), the clinician uses a hand-held device (not shown) to advance the stylet and the needle together to be close to the targeted and/or cancerous site within the targeted organ, wherein the stylet and the needle are aligned with an ideal insertion path (usually a straight path through the cancerous lesion site and/or the lesion center) displayed on the imagining guidance system. Second, the mechanism in the biopsy device triggers a high-speed linear advancement of the stylet, FIG. 2B. During this linear advancement, the tissue is cut and separated at the stylet tip. The separated tissue is then compressed and deformed based on the stylet geometry. Due to the forces acting on the bevel tip of the stylet during insertion and the tissue compression, along with the resultant asymmetric stylet beam shape created by the groove cutout, the stylet can be deflected significantly during this stage of the biopsy procedure. The outer needle is then advanced sequentially (FIG. 2C), following the deflected stylet insertion path, to cut the tissue and trap it inside the groove. FIG. 2D shows a cross-sectional view of the stylet and the needle wherein the sampled tissue fails to contain any cancerous tissue (lesion undersampling/missampling), leading to false negative results.

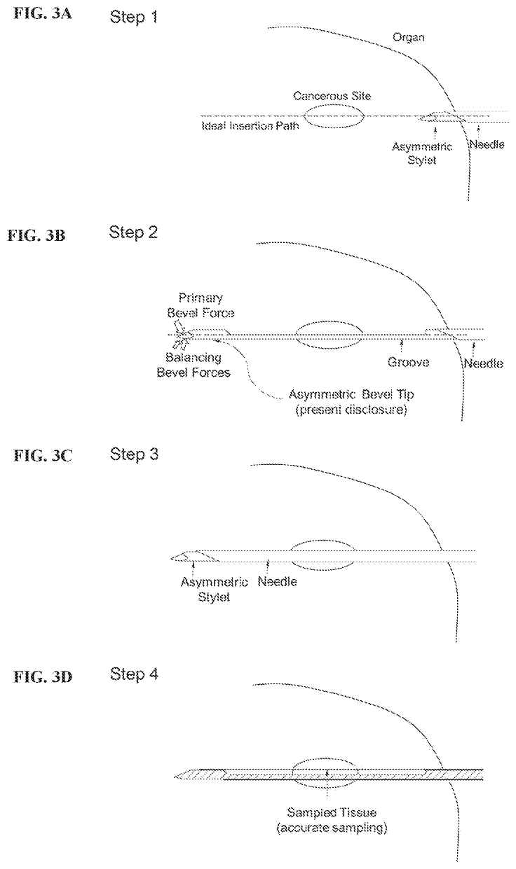

FIG. 3 shows the steps of a needle biopsy procedure using the stylet with a radially asymmetric stylet tip containing multiple bevels as in embodiments of the present disclosure. Similar to the aforementioned procedure, this stylet and needle are first positioned and aligned with the ideal insertion path through the cancerous site (FIG. 3A). The stylet and the needle are then advanced sequentially (FIGS. 3B and 3C) for tissue sampling. The stylet tip comprising a plurality of radially asymmetric bevels, including at least one primary bevel and at least one balancing bevel, improves the force and bending moment balance of the stylet to improve bending instability and achieve an insertion path that more closely follows the ideal straight insertion path. This is, for example, because the resulting bending forces and moments created by the balancing bevel(s) at least partially offset the resulting bending forces and moments created by the forces generated from the forces acting on the primary bevel and tissue compression. FIG. 3D shows a cross-sectional view of the stylet and the needle wherein the cancerous site is accurately sampled.

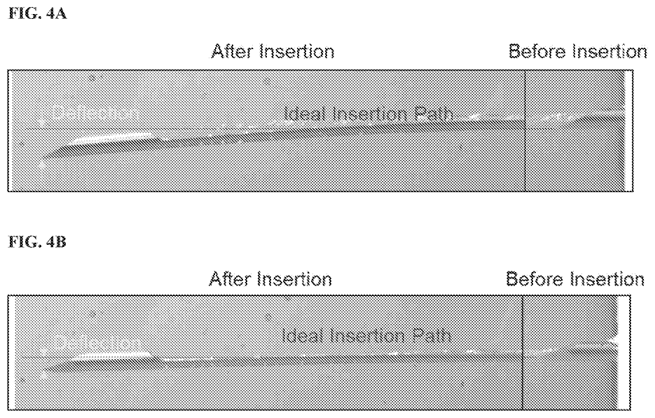

This is further demonstrated in FIG. 4. FIG. 4A shows a stylet with a single bevel (e.g., currently used design). This tip design causes the stylet to bend during the insertion into a healthy tissue-mimicking phantom, leading to accuracy issues where the needle deviates from the ideal straight insertion path (defined by a horizontal line starting from the initial cutting element (cutting point) before the insertion shown in FIG. 4A). FIG. 4B shows an asymmetric stylet tip with multiple bevels of embodiments of the present disclosure. This multi-bevel stylet tip design creates a more predictable, generally straight biopsy path compared to the commonly used stylet tip with a single bevel.

In some embodiments, as shown in Example 1 below, the biopsy device exhibits decreased deflection during deployment relative to a biopsy device lacking radially asymmetrical bevels with at least one primary and at least one balancing bevel (e.g., in some embodiments, the biopsy device exhibits less than 1 mm (e.g., less than 0.52 or 0.5 mm) deflection of a stylet with a diameter of 1 mm. In some embodiments, deflection is reduced by at least 50% (e.g., at least 55%, 60%, 65%, 70% or more) compared to an identical or similar device that lacks at least one primary and at least one balancing bevel on the stylet tip.

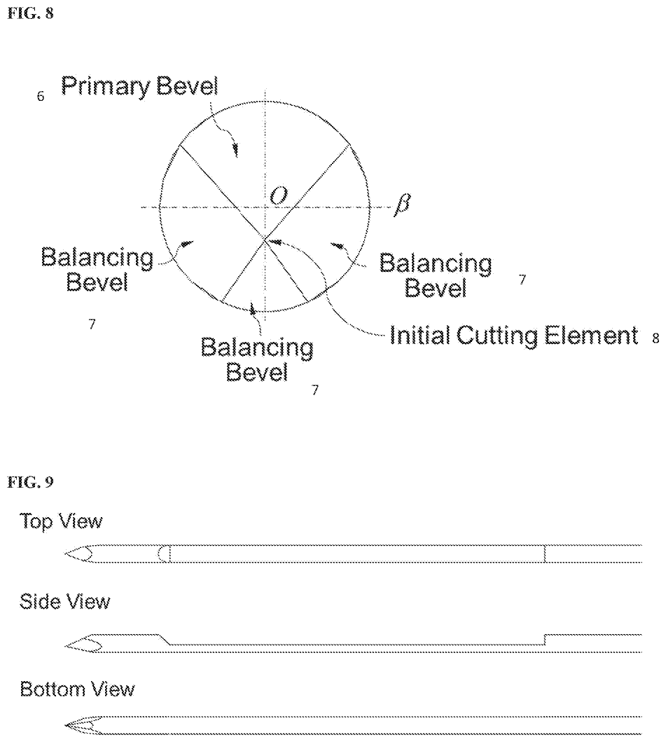

FIG. 5 shows an exemplary stylet 1. Referring to FIG. 5, stylet 1 comprises a stylet tip 2, and tissue storage groove 3 with groove face 5. Still referring to FIG. 5, stylet 1 further comprises a cylindrical body 4. Now referring to FIG. 9, shown are different perspective views of an exemplary stylet 1.

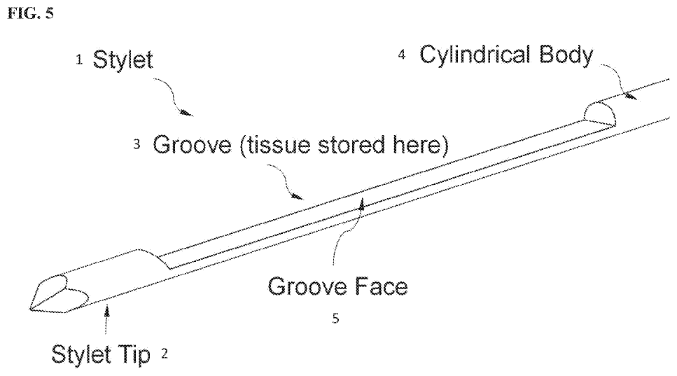

Now referring to FIG. 6, shown is a close-up view of stylet tip 2 of FIG. 5. Stylet tip 2 comprises a plurality of radially asymmetric bevels including at least one primary bevel 6 and at least one balancing bevel 7. Still referring to FIG. 6, the stylet tip 2 further comprises an initial cutting element 8. Still referring to FIG. 6, the embodiment shown comprises one primary bevel 6 and three balancing bevels 7.

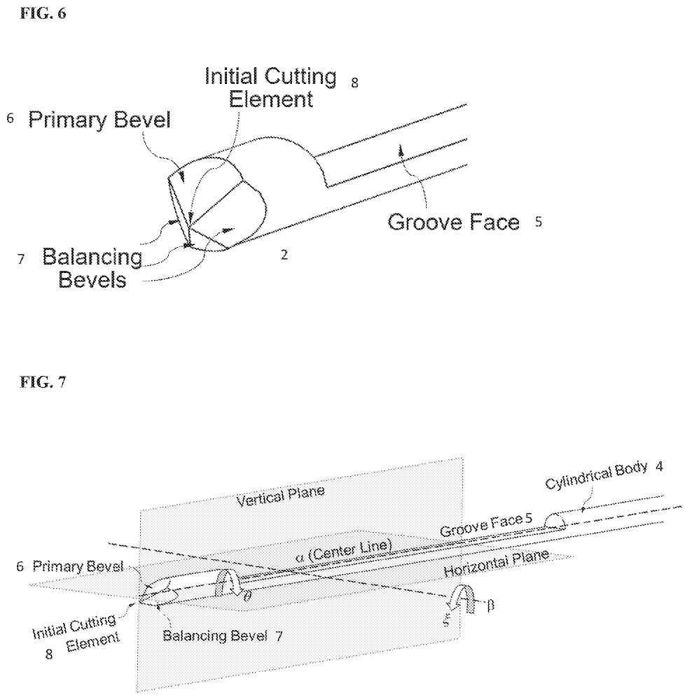

FIG. 7 shows a diagram of bevel radial orientation, θ, and bevel angle, ζ, for each bevel (including the primary bevel 6 and the balancing bevel(s) 7) on a radially asymmetric stylet tip. The bevels are radially asymmetric by the combination of θ and ζ of each bevel. These angles are defined by a stylet center line, α, which is the line through the center of the stylet cylindrical body 4, and line β, which is perpendicular to a and on the horizontal plane (the plane parallel or coincident to the groove face 5). In some embodiments, the balancing bevel 7 is created by rotating the stylet around the α line by θ relative to the primary bevel 6, and around the β line by ζ. Each balancing bevel 7 has its own θ. Each balancing bevel 7 has its own ζ, which can be the same or different from each other. Each balancing bevel 7 has its own ζ, which can be the same or different from the primary bevel 6. For example, in some embodiments, the bevel angles, ζ, are 10-25°. In some embodiments, the balancing bevels 7 are rotated by a θ of ±90-180° around the α line relative to the primary bevel 6. In the exemplary embodiment shown in FIGS. 7 and 8, the stylet tip comprises two side balancing bevels 7 rotated at θ=±90-130° and a bottom balancing bevel 7 rotated at θ=180° around the α line with respect to primary bevel 6. These balancing bevels induce bending forces and moments during tissue insertion which at least partially oppose the bending forces and moments generated by the primary bevel and the groove. This balances the bending instability during insertion for a more predictable and straighter insertion path. Such a predictable path increases the stylet deployment accuracy and the resultant tissue sampling accuracy in tissue biopsy procedures.

The present disclosure is not limited to a particular number of primary or balancing bevels. Exemplary configurations are shown in the figures described herein. In some embodiments, the balancing bevel number/angle/length/shape is varied to balance the bending forces caused by the tissue interaction during insertion. In some embodiments, the total area of the balancing bevels is generally larger than 20% of the area of the primary bevel(s) to provide sufficient forces to balance the bending instability. In some embodiments, the total area of the balancing bevels is larger than the area of primary bevel by 1.5-1.7 times.

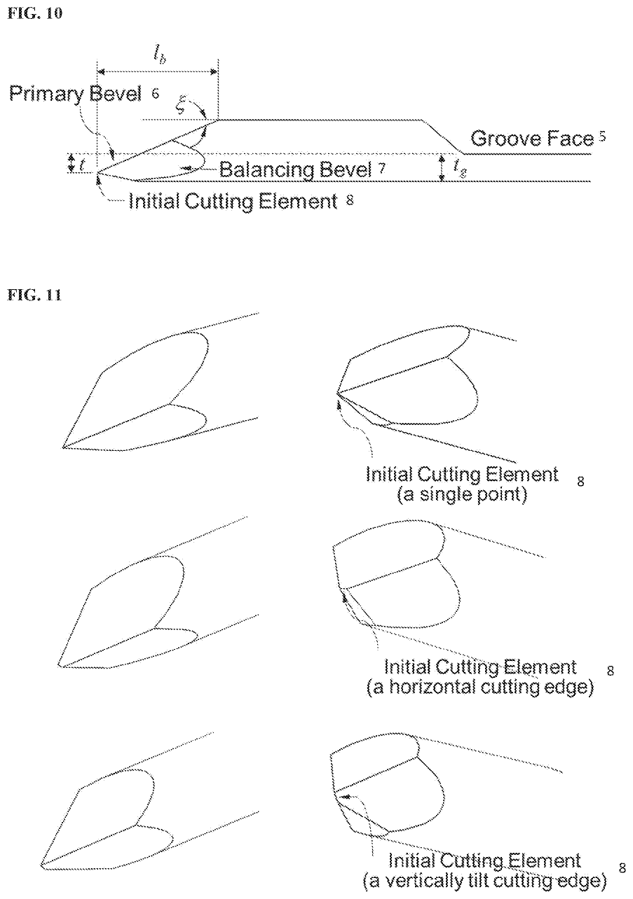

FIGS. 8 and 10 show a diagram of a head-on (FIG. 8) and side view (FIG. 10) of an exemplary stylet tip. Shown is the location of the primary bevel 6 and the initial cutting element 8. The primary bevel 6 and balancing bevels 7 are radially asymmetric about center point O. In some embodiments, the bevels including the primary bevel(s) 6 and the balancing bevel(s) 7 are asymmetric relative to β line (defined above). The primary bevel 6 comprises a bevel angle ζ, and a bevel length, l_{b} (FIG. 10). Each balancing bevel also has its own bevel angle ζ, and a bevel length, l_{b}, which can be different from each other (also can be different from that of primary bevel). Numbers, angles and the lengths of the bevels depend on the groove geometry (e.g., groove length, groove depth, opening angles, etc.). For example, a shallow groove may need shorter/smaller balancing bevels to counter the bending forces while a deeper groove may need longer/larger balancing bevels to provide more supporting forces to resist the bending. The bevels, including the primary bevel 6 and the balancing bevels 7 converge to form the initial cutting element 8. In some embodiments, the initial cutting element is below or at least partially aligned with the tissue groove face. In the exemplary embodiment shown in FIG. 10, the initial cutting element 8, which is a single cutting point, is below the groove face 5 by a distance t. The value of t is determined by the stylet groove thickness t_{g}. In some embodiments, the initial cutting element 8 is below the groove face 5 by 10-70% of the t_{g}. In some embodiments, the initial cutting element 8 is below the groove face 5 by 0.003-0.011″ for a stylet with the t_{g} of 0.017″. Such an arrangement ensures that the tissue separation occurs below the groove face 5 to allow the tissue to fill in the groove for adequate tissue sampling or at least equivalent tissue sampling when compared to the existing stylet tip with a single bevel.

In some embodiments, the primary and balancing bevels converge to form initial cutting element 8. The present disclosure is not limited to particular initial cutting element 8. Exemplary cutting elements are shown in FIG. 11 and include but are not limited to, a point (top view of FIG. 11), a horizontal cutting edge (middle view of FIG. 11), and a vertically tilted cutting edge (bottom view of FIG. 11). In some embodiments, both ends of the cutting element 8 (vertical tilt edge with respect to groove face) are below or at least partially aligned with the tissue groove face 5.

Now referring to FIG. 12, shown are additional stylet tip designs. Referring to FIG. 12A, shown is a stylet tip with one primary bevel 6 and one balancing bevel 7 and an initial cutting element 8 of a horizontal cutting edge. Now referring to FIG. 12B, shown is an embodiment with one primary bevel 6 and two balancing bevels 7. Now referring to FIG. 12C, shown are a plurality of exemplary different combinations of initial cutting elements and bevel angles and lengths.

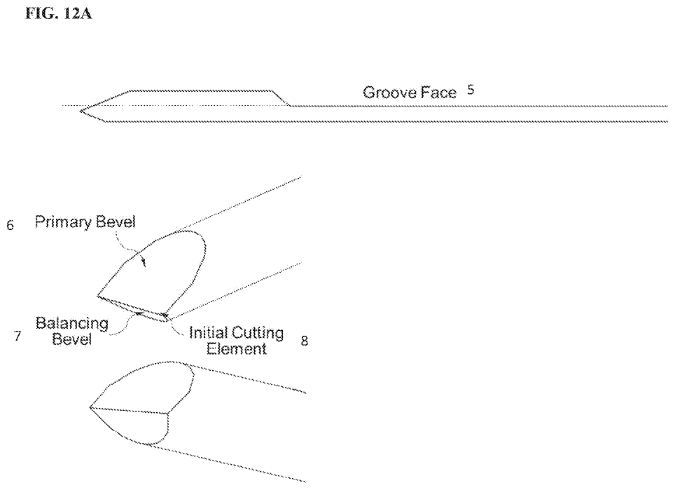

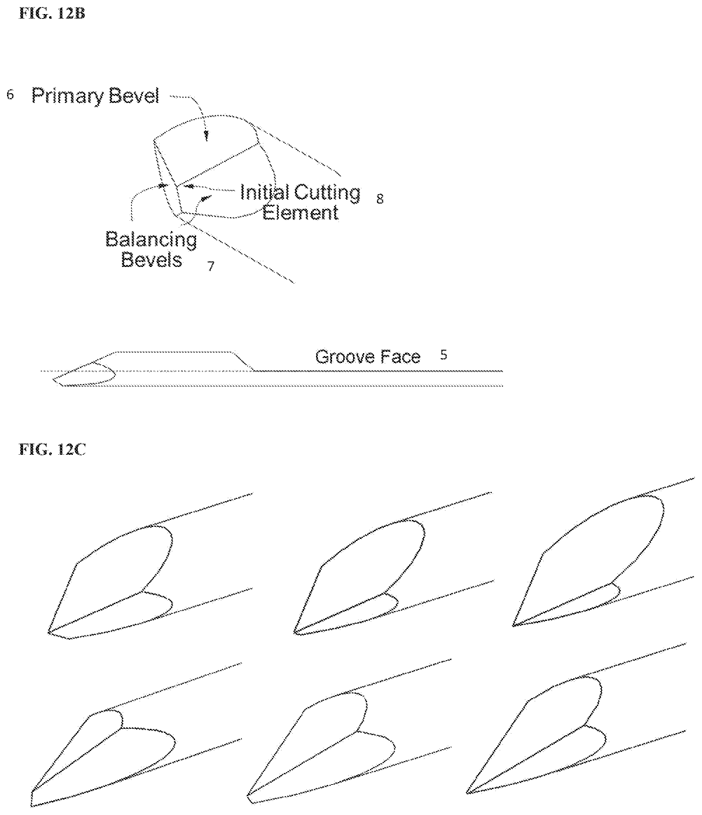

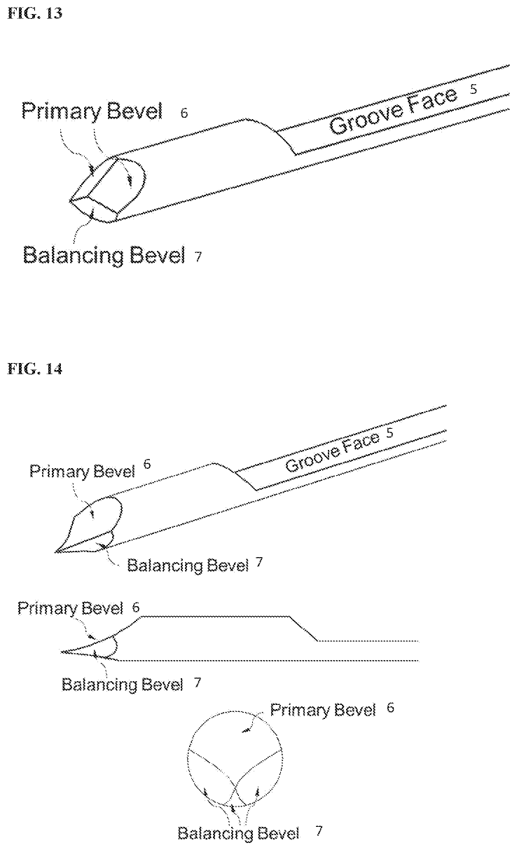

Now referring to FIG. 13, shown is an isometric view of an exemplary stylet tip with two primary bevels 6 and one balancing bevel 7.

Now referring to FIG. 14, shown are isometric, side, and front views of an exemplary stylet tip with one concave shaped primary bevel 6 and three concave shaped balancing bevels 7.

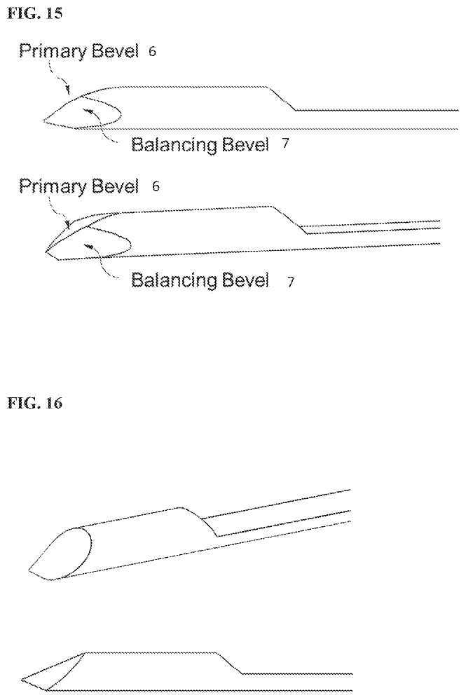

Now referring to FIG. 15, shown are isometric and side views of an exemplary stylet tip with one convex shaped primary bevel 6 and two convex shaped balancing bevels 7.

Now referring to FIG. 16, shown are isometric and side views of an exemplary stylet tip with continuous primary and balancing bevels.

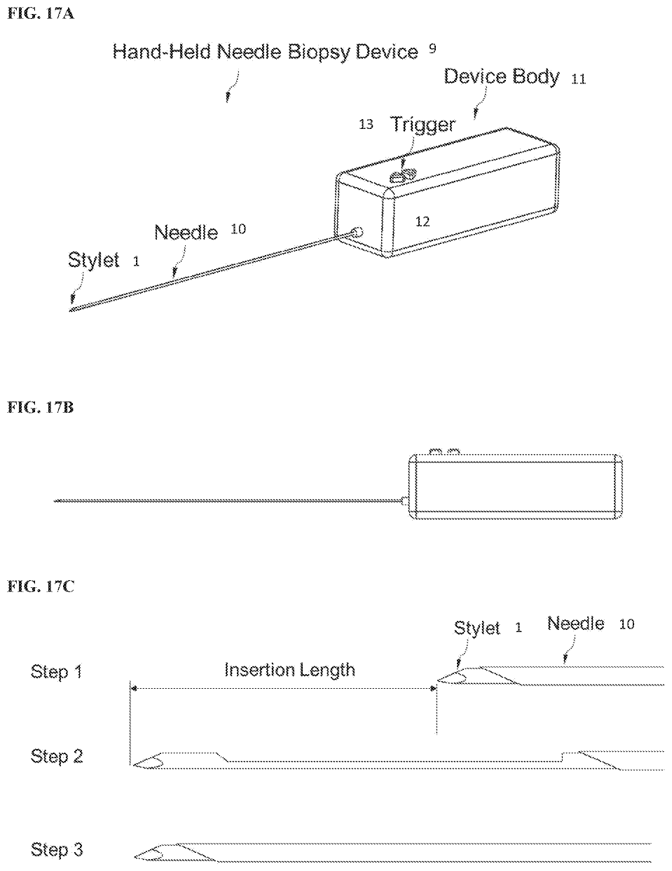

Now referring to FIGS. 17A and 17B, shown is a hand-held needle biopsy device 9 of an exemplary embodiment of the present disclosure wherein device comprises a stylet 1, a hollow needle 10 and a device body 11 with a deployment component 12 that advances the stylet 1 and needle 10 sequentially to perform a tissue biopsy. Examples of deployment component 12 include but are not limited to spring, pneumatic source, hydraulic source, and motor. The advancements of the stylet 1 and the needle 10 are activated by one or more deployment components 12 on the device body 11. In some embodiments, the deployment component 12 is activated by a switch or trigger 13. Examples of trigger 13 include button(s) or moveable lever(s) that can activate the stylet 1 and needle 10 to advance sequentially (FIG. 17C) with a set insertion length at a set or arbitrary time period depending on the user.

The biopsy devices described herein find use in a variety of biopsy procedures. In some embodiments, the biopsy devices find use in obtaining samples from a tissue suspected of being cancerous or comprising a different pathology. The biopsy devices described herein find use in a variety of different tissues (e.g., including but not limited to, liver, lung, kidney, breast, and prostate tissues).

### EXPERIMENTAL

### Example 1

This example describes a comparison of deflection and biopsy yield of a single bevel stylet tip versus a radially asymmetric multi-bevel stylet tip with one primary bevel and three balancing bevels, both installed on the SelectCore™ Variable Throw Biopsy Device (by Inrad, Grand Rapids, Mich., USA). FIGS. 5-10 show the geometry of the asymmetric stylet used. The stylets both had identical gauge size (18 gauge, stylet diameter of 1 mm), groove width (1 mm), groove length (21.3 mm), groove depth (0.43 mm) and groove position (5.15 mm from tip). The single bevel has a θ=0° and ζ=23.5°. The radially asymmetric multi-bevel stylet tip has a primary bevel at θ=0° and ζ=23.50 a balancing bevel at θ=180 and ζ=12°, θ=+110° and ζ=12% and θ=−110° and ζ=12° with t=0.23 mm.

FIG. 18 and Table 1 show the results of a needle insertion experiment using prostate tissue-mimicking phantom made of polyvinyl chloride (W. Li, et al. Med. Phys. 43 (2016) 5577-5592) which is designed to mimic healthy prostate tissue. Each stylet was inserted into the phantom with N=30. The average deflection of the single bevel tip was 1.33 mm. In contrast, the asymmetric tip exhibited an average deflection of 0.52 mm (61% reduction in deflection).

TABLE 1

| | | Asymmetric |
|---|---|---|
| | Single Bevel | Multi-Bevel |
| | Stylet Deflection | Stylet Deflection |
| | [mm] | [mm] |
| | 1.49 | 0.61 |
| | 1.60 | 0.48 |
| | 1.69 | 0.56 |
| | 1.62 | 0.62 |
| | 1.61 | 0.64 |
| | 1.63 | 0.75 |
| | 1.50 | 0.93 |
| | 1.71 | 0.53 |
| | 1.61 | 0.92 |
| | 1.59 | 0.72 |
| | 1.60 | 0.68 |
| | 1.37 | 0.26 |
| | 1.13 | 0.28 |
| | 1.19 | 0.52 |
| | 1.24 | 0.47 |
| | 1.25 | 0.55 |
| | 1.24 | 0.42 |
| | 1.26 | 0.51 |
| | 1.30 | 0.61 |
| | 1.15 | 0.63 |
| | 1.13 | 0.45 |
| | 1.23 | 0.47 |
| | 1.29 | 0.43 |
| | 1.09 | 0.58 |
| | 1.24 | 0.46 |
| | 1.32 | 0.62 |
| | 1.11 | 0.42 |
| | 0.93 | 0.33 |
| | 1.22 | 0.49 |
| | 1.27 | 0.27 |
| | 1.05 | 0.31 |
| | 1.04 | 0.37 |
| | 1.16 | 0.43 |
| Averaged Deflection [mm] | 1.33 | 0.52 |
| Standard Deviation [mm] | 0.22 | 0.16 |
| Reduction in Deflection | N/A | 61 |
| [%] | | |

Table 2 shows the results of a tissue yield experiment using chicken breast to mimic prostate tissue with N=30. The average sample weight of the single beveled tip (0.06 g) and multi-bevel asymmetric stylet tip (0.05 g) were similar.

TABLE 2

| | | | Asymmetric | |
|---|---|---|---|---|
| | Single Bevel Stylet | | Multi-Bevel Stylet | |
| | | Weight | | Weight |
| | | (average for | | (average for |
| | Length | 10 samples) | Length | 10 samples) |
| | [mm] | [g] | [mm] | [g] |
| | 10.39 | 0.051 | 9.70 | 0.053 |
| | 10.15 | | 8.62 | |
| | 9.40 | | 9.72 | |
| | 10.85 | | 9.59 | |
| | 11.73 | | 11.05 | |
| | 9.49 | | 11.60 | |
| | 11.4 | | 8.68 | |
| | 8.64 | | 11.50 | |
| | 9.11 | | 10.23 | |
| | 10.01 | | 9.30 | |
| | 12.99 | 0.060 | 10.20 | 0.050 |
| | 11.99 | | 9.59 | |
| | 12.00 | | 11.33 | |
| | 12.12 | | 9.13 | |
| | 8.49 | | 11.06 | |
| | 10.88 | | 9.64 | |
| | 10.76 | | 8.76 | |
| | 12.30 | | 8.91 | |
| | 9.74 | | 10.24 | |
| | 9.95 | | 10.40 | |
| | 10.43 | 0.057 | 9.51 | 0.052 |
| | 10.52 | | 11.06 | |
| | 10.25 | | 11.30 | |
| | 11.11 | | 9.37 | |
| | 8.08 | | 12.4 | |
| | 10.19 | | 9.17 | |
| | 9.13 | | 9.57 | |
| | 10.57 | | 10.28 | |
| | 11.46 | | 10.35 | |
| | 9.80 | | 11.40 | |
| Averaged | 10.46 | 0.056 | 10.12 | 0.052 |
| Length/Weight | | | | |
| [mm/g] | | | | |
| Standard | 1.20 | 0.0046 | 1.00 | 0.0015 |
| Deviation | | | | |
| [mm/g] | | | | |

Table 3 shows the tissue sampling results of an experiment using turnip to mimic cancerous prostate tissue. Three devices of each design were used. Results of the sampling length for each design were comparable. FIG. 19 shows the stylet bending results during the turnip sampling test. The single bevel tip was permanently deformed after one deployment into the turnip with significant deflection. In contrast, the asymmetric multi-bevel tip described in this example maintained a nearly straight profile even after 5 repeated deployments into the turnip.

TABLE 3
Results of tissue sampling length

| | Single Bevel Stylet | Asymmetric Multi-Bevel Stylet |
|---|---|---|
| | Tissue Length [mm] | |
| Device 1 | 19.46 | 17.26 |
| Device 2 | 17.15 | 18.91 |
| Device 3 | 18.30 | 18.10 |

In conclusion, this example demonstrates that a multi-beveled radially asymmetric stylet tip is capable of achieving clinically equivalent biopsy yield volume, a 61% reduction in stylet deflection, and a more resilient stylet for penetrating cancerous tissue.

### Example 2

This example compares stylet deflection and tissue sampling quality between single and multi-bevel stylet tip biopsy devices. This example demonstrates that an asymmetric multi-bevel stylet (present disclosure) with multiple balancing bevels at the tip and an initial cutting element below the groove face reduces stylet deflection while maintaining sufficient/equivalent tissue sampling compared to the existing single bevel stylets.

During biopsy procedure, an asymmetric multi-bevel stylet is first fired at high speed (about 4 m/s) and subjected to the cutting, primary bevel face, and balancing bevel face forces at the tip as well as the tissue pressure and friction force on the needle. Those balancing bevels are important to keep the initial cutting element below the groove face while generating the combined upward face force to balance the downward bending caused by the combined top face force and tissue pressure in the groove, resulting the low stylet bending moment and deflection. Next, the outer needle is then fired to cut and store the tissue inside the stylet groove. With the initial cutting element (a cutting point in this example) below the groove face, the tissue is filled inside most of the groove and needle can cut and acquire a long tissue sample.

In this Example, the stylet deflection and tissue sampling of a currently used single-bevel and three asymmetric multi-bevel tip geometries in tru-cut biopsy are quantified and compared. The needle deflection is experimentally measured in optically transparent tissue-mimicking phantoms and analyzed by image processing. The length and weight of sampled tissue in biopsy of ex-vivo chicken breast tissue are investigated. Finally, the evaluation of the multi-bevel trucut needle biopsy device on human cadaver prostate is performed.

### Materials and Methods

### Needle Tip Geometry

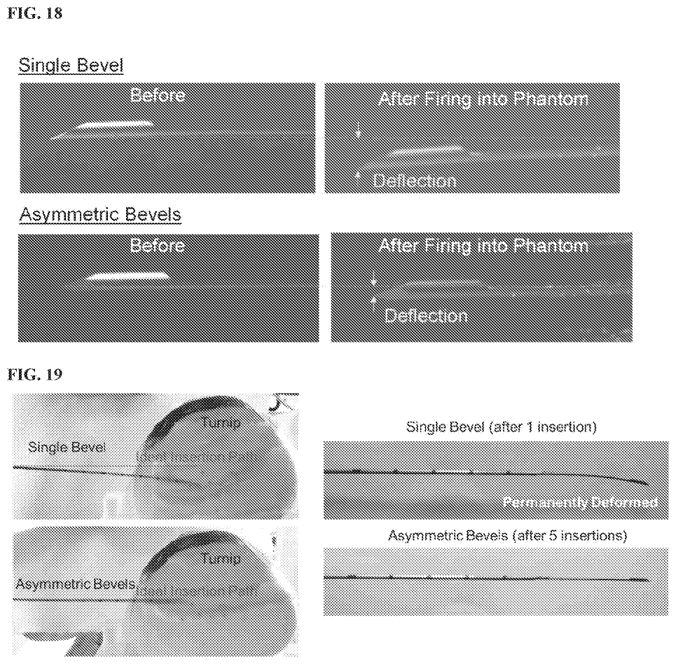

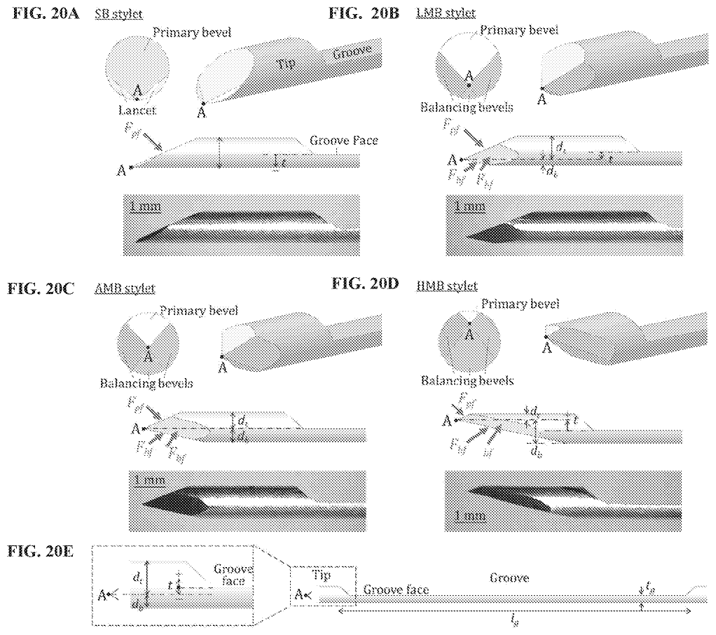

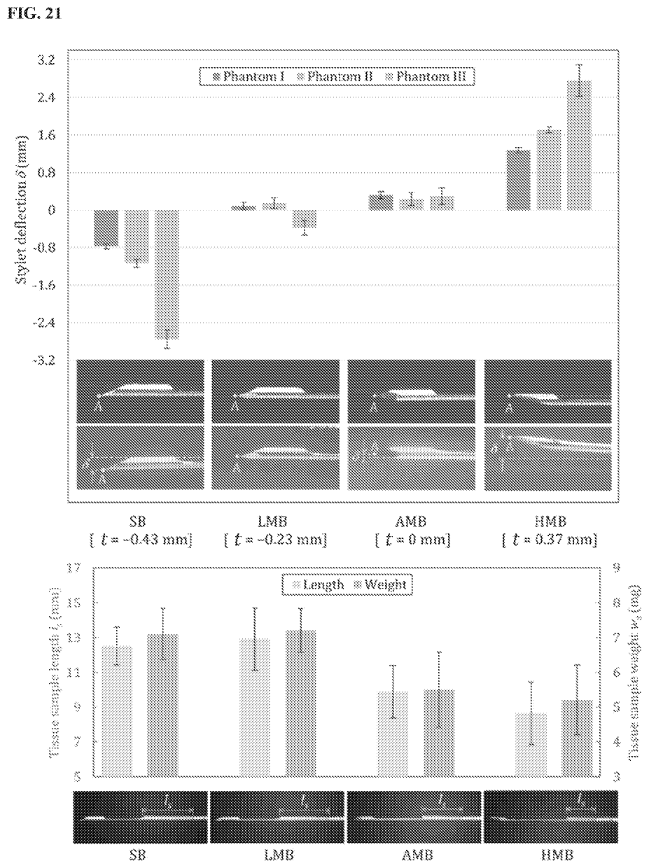

The single-bevel (SB) stylet, as shown in FIG. 20A, and three asymmetric multi-bevel stylets, as shown in FIGS. 21B-D, were investigated in this study. The three multi-bevel stylets have four facets: one primary bevel (on the same side of the groove face) and three balancing bevels with one at the bottom (θ=180°) and two on the both sides (θ=±110°). The force on the primary bevel face is F_{pf} and on the balancing bevels are denoted as F_{bf}. The three types of multi-bevel stylets shown in FIGS. 21B-D are denoted as the low multi-bevel (LMB), aligned multi-bevel (AMB), and high multi-bevel (HMB) with the initial cutting element (a single cutting point A in this Example) lower than, aligned with, and higher than the groove face, respectively. The LMB stylet represents an embodiment of the present disclosure. The AMB and HMB stylets are not the embodiments of the present disclosure but used to emphasize the importance of having an initial cutting element below the groove face for adequate tissue sampling.

The distance from the initial cutting point A to the groove face t, as defined in FIG. 20E, is negative, zero, and positive for the LMB, AMB, and HMB stylet, respectively. The effect of t on stylet deflection and tissue sampling length and weight is studied using LMB, AMB, and HMB stylets. FIG. 20E defines the stylet groove length l_{g} and thickness t_{g}. The distance between point A and the bottom edge of the stylet is d_{b}. The sum of d_{b} and dₜ (the distance between point A and the top edge of the stylet) is the stylet diameter. Both d_{b} and dₜ have positive values.

The shape, features, forces on four facets, and optical microscopy images of the SB, LMB, AMB, and HMB stylet tip are shown in FIGS. 20A-D and discussed as follows:
SB stylet: The SB stylet, as shown in FIG. 20A, has a single primary bevel face (on the same side of the needle groove) and two small side lancets at the tip. These two lancets (not the balancing bevels described in this Example) create a sharp tip point A (Yang, B. L. et al., J. Manuf. Sci. Eng. 135 (2013) 041010) aiming to increase the tip sharpness. The tissue is cut and separated at point A, contacts the primary bevel face, and generates the downward forces, F_{pf}, which bend the stylet during insertion in biopsy. The initial cutting tip point A is below the groove face (t<0).
LMB stylet: The LMB stylet, as shown in FIG. 20C, has three balancing bevels with one at the bottom (θ=180°) and two on the both sides (θ=±110°) generating the upward face forces F_{bf} to balance the downward bending moments caused by the top face F_{pf} and tissue pressure. Similar to the SB stylet, the LMB stylet has the tip point A below the groove face (t<0).
AMB stylet: The AMB stylet, as shown in FIG. 20D and compared to LMB stylet, has larger bottom and side bevels for the larger combined upward forces F_{bf}, aiming to reduce the downward stylet deflection.
HMB stylet: Like LMB and AMB stylets, the HMB stylet, as shown in FIG. 20E, has the bottom bevel face much larger than the other three faces. This greatly increases an overall larger F_{bf} to deflect the needle upward during the insertion.

In this Example, four stylet tip geometries and the groove were fabricated by computer numerical control grinding using a 18-gauge (1 mm diameter) AISI 304 stainless steel rod. In the fabrication, the steel rod was first tilted by a bevel angle of 23.5° to grind a primary bevel facet (for the SB stylet). The lancets for the SB stylet were added onto this bevel face (Yang et al., supra). For LMB, AMB, and HMB stylets, the rod was then tilted to a second bevel angle of 12° and rotated around the needle centerline axis by 180° and ±110° from the primary bevel facet to create the bottom and two side bevel facets, respectively, as the balancing bevels at the tip. The ground amount for each bevel facet was determined by the t and d_{b} at the needle tip. The SB stylet has t=−0.43 mm and d_{b}=0 mm. The LMB stylet has t=−0.23 mm and d_{b}=0.2 mm. The AMB stylet has t=0 mm and d_{b}=0.43 mm. The HMB stylet has t=0.37 mm and d_{b}=0.8 mm. Finally, the rod was tilted back to 0° to grind the needle groove with the l_{g}=22 mm and t_{g}=0.43 mm. All four stylets had the same groove geometry.

### Tissue-Mimicking Phantoms

Tissue-mimicking phantoms made of polyvinyl chloride (PVC) were used as the surrogate for soft tissue in the needle deflection experiments. PVC is a common tissue-mimicking material and can be fabricated with the hardness and needle insertion properties similar to in-vivo prostate tissues (W. Li, et al., Med. Phys. 43 (2016) 5577-5592; D. Li, et al., in: Vol. 4 Bio Sustain. Manuf., ASME, 2017: p. V004T05A010). The softener, PVC polymer (both by M-F Manufacturing, Ft. Worth, Tex., USA), and mineral oil (by W.S. Dodge Oil, Maywood, Calif. USA) were blended together to create the phantom material with the targeted hardness (W. Li, et al., Med. Phys. 43 (2016) 5577-5592). In this study, the transparent PVC phantom with 100 mm in length, 80 mm in width, and 30 mm in height, was fabricated. Each phantom has a uniform hardness to study needle deflection in a specific material property. Three PVC phantoms, namely Phantoms I, II and, III, were built to mimic the soft tissue surrounding prostate, outer soft layer of prostate, and inner hard core of the prostate with Shore OOO-S hardness of 23, 34 and 55, respectively. These hardness values were determined based on clinician's haptic feedback for the hardness of a specific organ.

### Stylet Deflection Experimental Setup

A commercial spring-loaded needle biopsy device (SelectCore Variable Throw Biopsy Device by Inrad, Kentwood, Mich., USA) was used to perform the stylet insertion with a 25 mm firing length for both stylet and needle. Both stylet and needle were installed on the biopsy device and supported by a prostate biopsy guide (Endfire Biopsy Guide by BK Medical, Peabody, Mass., USA). The biopsy guide had a plastic semi-cylindrical body for the ultrasound probe guide and a metal tube for the stylet/needle guide. The biopsy guide was fixed to position the stylet and support it to avoid buckling during needle insertion. In the experiment, the biopsy guide was used to place the stylet at the surface of the phantom for insertion. The biopsy device fired only the stylet at a high speed (about 4 m/s) to have a clear view of the stylet deflection. A high-speed camera (Model 100K by Photron, San Diego, Calif., USA) with 1024×1024 pixel resolution and a 5.6× magnification was used to capture the images of stylet tip before and after the insertion to measure the stylet deflection.

To acquire the baseline tip position without deflection, the stylet was first inserted without the phantom. The stylet was then advanced by the biopsy device into the transparent phantom. The stylet deflection δ was calculated as the vertical distance (relative to the insertion direction) between the final tip locations with and without the phantom. Ten insertions of each stylet tip types (SB, LMB, AMB, and HMB) were performed for each phantom (Phantoms I, II, and III) at different locations in the phantom. A total of 120 stylet insertion tests were performed. The images were analyzed using Matlab (by MathWorks, Natick, Mass., USA) to identify the stylet tip locations and quantify the deflections.

### Ex-Vivo Tissue Sampling Test

The tissue sampling amount for four stylet tip types (SB, LMB, AMB, and HMB) was quantified in the tru-cut needle biopsy tests using ex-vivo chicken breast tissue. The stylet and outer needle were sequentially fired by the biopsy device (same as that of stylet deflection experiments) into the ex-vivo tissue fixed on a platform for tissue sampling. For each type of stylet tip, ten insertions were performed at different locations of the ex-vivo tissue. A total of 40 needle biopsies were conducted. The length of each tissue sample lₛ was measured using a digital caliper with the sample staying on the stylet groove after biopsy. The tissue sample was then removed from the groove to measure the weight wₛ using a digital scale (Gemini-20 by American Weigh Scales, Cumming, Ga., USA). The stylet and needle were rinsed and dried before the next biopsy.

### Cadaver Prostate Tissue Sampling Test

The tissue sampling test on cadaver prostate tissue was conducted to evaluate the biopsy performance on human tissue for SB and LMB stylets (both with t<0). The tissue was refrigerated for storage and recovered at room temperature prior to the test. The prostate has a size of about 45 mm in diameter with part of the bladder wall and the surrounding soft tissues. In this test, the tissue surrounding the prostate was fixed to maintain the in-vivo weakly supported condition for prostate biopsy. Five insertions were performed at different locations of the prostate for the SB and LMB stylets. A total of 10 needle biopsies were conducted in the cadaver prostate. The length of each tissue sample was measured using a digital caliper with the sample staying on the stylet groove. After each measurement, the stylet and needle were rinsed and dried to remove the tissue before the next insertion.

### Statistical Analysis

One-way analysis of variance (ANOVA) tests were performed to calculate the statistical significance among the experimental data of stylet deflection (in three phantoms) and the lengths and weights of tissue samples (in chicken breast and cadaver prostate) for SB, LMB, AMB, and HMB stylets. Each stylet has ten data points for each measured variable. A total of 40 data points was used in each ANOVA test. The mean values in each experiment of any two of the four stylets were compared (pairwise comparisons) to calculate the p values with Bonferroni correction at 95% confidence level.

### Results

### Stylet Deflection and Tissue Sampling Results

FIG. 21 and Tables 4-6 summarizes the mean values of stylet deflection δ in three phantoms (top), and tissue sample length lₛ and weight wₛ of chicken breast tissue (bottom) with the error bars representing the standard deviations for the SB, LMB, AMB, and HMB stylets. Two images of stylet tip point A in Phantom H experiment are shown in FIG. 21. The top image shows the needle tip location before the insertion with the yellow dashed line marked as the insertion path without stylet deflection. The bottom image shows the stylet with deflection after inserting into the phantom. The optical microscopy images of the tissue samples on the stylet groove are also presented. Table 4 shows the p values in ANOVA tests for each pairwise comparison (any two of SB, LMB, AMB, and HMB needles) of δ, lₛ, and wₛ.

TABLE 4
Results of p values in ANOVA tests for the pairwise comparisons
of δ, lₛ, and wₛ for SB, LMB, AMB, and HMB stylet s.

| | | Stylet deflection δ | | | Tissue sampling | |
|---|---|---|---|---|---|---|
| | | Phantom | Phantom | Phantom | Length | Weight |
| | | I | II | III | lₛ | wₛ |
| SB | LMB | * | * | * | 1.000 | 1.000 |
| | AMB | * | * | * | 0.004 | 0.002 |
| | HMB | * | * | * | * | * |
| LMB | AMB | * | 0.457 | * | 0.001 | 0.001 |
| | HMB | * | * | * | * | * |
| AMB | HMB | * | * | * | 0.510 | 1.000 |

(* p < 0.001)

The SB stylet (t=−0.43 mm) had a large δ of −0.78, −1.14, and −2.75 mm in Phantoms I, II, and III, respectively, and also yielded a long lₛ of 12.5 mm with wₛ of 7.1 mg. The downward force on the primary bevel face significantly deflected the stylet, as shown in FIG. 21. The stylet deflection correlates positively with the hardness of the phantom material due to the increased stylet insertion forces. This resulted in the largest downward Sin all three phantoms among four stylet s (all with pairwise p<0.001 as shown in Table 4). The magnitude of δ was similar to the clinically measured stylet/needle defection (using ultrasound images) with a median value of 1.77 mm in prostate biopsy (Halstuch, J. et al., J. Endourol. 32 (2018) 252-256). Since the initial cutting point is below the groove face (t<0), the SB stylet allowed the tissue to fill the groove and enabled a long (over 12 mm) tissue sample, as shown in FIG. 21. However, such tissue contact generated tissue pressure on the groove face, which further aggravated the stylet deflection.

The LMB stylet (t=−0.22 mm) had a low δ of 0.09 (almost 0), 0.15, and −0.37 mm in Phantoms I, II, and III, respectively, while maintaining a long lₛ of 12.9 mm with wₛ of 7.2 mg. Compared to the SB stylet, the magnitude of δ was much lower in all three phantoms (p<0.001, Table 4). This indicated that the LMB stylet can potentially achieve better deployment accuracy with lower deflection in a biopsy procedure. The balancing bevel faces generated the upward face forces, which balance the downward bending moments caused by the primary face force and tissue pressure on the groove face. This resulted in a slightly upward Sin Phantoms I and II, as shown in FIG. 21. In Phantom III, the δ became downward because of the increased primary face force and tissue pressure caused by the high hardness of Phantom II. Since t<0, as shown in FIG. 21, the LMB stylet also had high lₛ and wₛ, which are equivalent to that of the SB stylet (p=1.000, non-significant, as indicated in Table 4). The LMB stylet enabled both accurate needle insertion and tissue sampling in biopsy and was demonstrated to be an ideal tip design (present disclosure).

The AMB stylet (t=0 mm) had an upward δ of 0.32, 0.24, and 0.30 mm in Phantoms I, II, and III, respectively, and a lₛ of 9.9 mm with wₛ of 5.5 mg. Compared to the LMB stylet, the AMB stylet had a larger upward δ for three phantoms (p<0.001 in Phantoms I and III, p=0.457 in Phantom II). The AMB stylet, compared to the LMB stylet, had larger bottom and side balancing bevel faces and generated the upward forces to deflect the stylet upward, as shown in FIG. 21. The δ was almost identical in all three phantoms for AMB stylet. Since t=0 mm, the location of the initial cutting point was higher than that of SB and LMB stylets, resulting in lower lₛ and wₛ (p<0.005 with both SB and LMB stylets).

The HMB stylet (t=0.37 mm) had the large upward δ of 1.27, 1.71, and 2.76 mm in Phantoms I, II, and III, respectively, and the short lₛ of 8.6 mm with wₛ of 5.2 mg. Since the bottom balancing bevel face was much larger than the other three faces, the combined balancing face forces significantly deflected the stylet upward, as shown in FIG. 21, with all pairwise p<0.001. The magnitude of δ also increased with the hardness of phantom material, the same trend observed in the δ of SB stylet. The HMB stylet has the lowest lₛ and wₛ among all four needles (p<0.001 with both SB and LMB stylet s, p=0.510 with the AMB stylet) due to t>0.

In summary, the LMB stylet is an ideal design enabling both low stylet deflection by self-balancing the stylet bending moments and high tissue sampling (lₛ and wₛ) with t<0 (below the groove face). The AMB stylet also had low needle deflection while the tissue sampling was limited due to t=0 (aligned with the groove face). The SB stylet in current tru-cut biopsy device (t<0) yielded high lₛ and wₛ but had a large downward deflection during stylet insertion as the result of tip geometry with a single primary bevel. Finally, the HMB stylet caused large upward deflection and greatly reduced lₛ and wₛ as a result of the high cutting point location (t>0).

### Cadaver Prostate Test Results

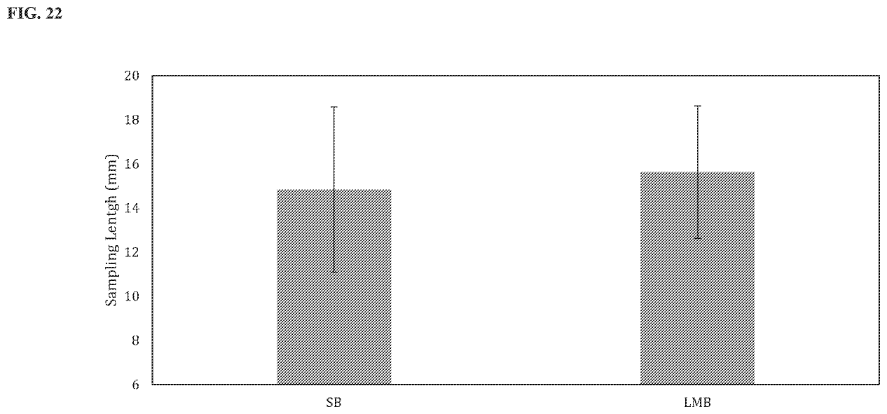

FIG. 22 summarizes the results of tissue sampling length in the cadaver prostate tests for SB and LMB stylets (both with t<0). The average sample length was 14.8 and 15.6 mm for the SB and LMB stylets, respectively. The LMB stylet had an equivalent tissue sampling length compared to that of the SB stylet (p=0.676). The capability of tissue sampling on human cadaver prostate for the LMB stylet biopsy device was confirmed.

### CONCLUSIONS

This study revealed two important design criteria for ideal stylet in tru-cut needle biopsy: 1) the initial cutting element should be below the stylet groove face to ensure high tissue sampling and 2) the multi-bevel stylet tip geometry, which can have balancing bevel faces generating upward forces while maintaining the low cutting point, is used to balance the bending moments during the insertion and enable low stylet deflection. In this study, the LMB stylet demonstrated the lowest stylet deflection (with up to 88% reduction in magnitude compared to SB stylet) and long tissue sampling among SB, LMB, AMB, and HMB stylets. The capabilities of improved stylet/needle deployment accuracy and tissue sampling on human tissue for a needle biopsy device with a LMB stylet have also been confirmed. Results from this Example have broad applications for various biopsy procedures as well as other procedures requiring accurate needle insertion.

TABLE 5
Stylet deflection δ results in Phantom
I, II and III for SB, LMB, AMB, and HMB stylets.

| | Stylet deflection δ [mm] | | | | | | | | | | | |
|---|---|---|---|---|---|---|---|---|---|---|---|---|
| | Phantom I | | | | Phantom II | | | | Phantom III | | | |
| | SB | LMB | AMB | HMB | SB | LMB | AMB | HMB | SB | LMB | AMB | HMB |
| | −0.77 | 0.03 | 0.32 | 1.32 | −1.08 | 0.07 | 0.36 | 1.82 | −2.61 | −0.19 | 0.35 | 2.99 |
| | −0.89 | 0.03 | 0.40 | 1.36 | −1.02 | 0.17 | 0.28 | 1.72 | −2.80 | −0.44 | 0.58 | 3.24 |
| | −0.72 | 0.11 | 0.23 | 1.20 | −1.20 | 0.22 | 0.02 | 1.82 | −2.61 | −0.31 | 0.08 | 2.68 |
| | −0.74 | −0.04 | 0.34 | 1.32 | −1.08 | 0.26 | 0.42 | 1.69 | −2.57 | −0.22 | 0.13 | 2.61 |
| | −0.73 | 0.21 | 0.30 | 1.22 | −1.03 | 0.21 | 0.11 | 1.65 | −2.73 | −0.33 | 0.24 | 3.06 |
| | −0.75 | 0.07 | 0.35 | 1.26 | −1.19 | 0.27 | 0.17 | 1.67 | −2.81 | −0.68 | 0.29 | 2.67 |
| | −0.77 | 0.07 | 0.36 | 1.33 | −1.25 | 0.10 | 0.26 | 1.65 | −2.74 | −0.56 | 0.49 | 2.45 |
| | −0.82 | 0.10 | 0.30 | 1.31 | −1.26 | 0.22 | 0.25 | 1.66 | −3.14 | −0.45 | 0.05 | 2.86 |
| | −0.78 | 0.15 | 0.16 | 1.24 | −1.11 | −0.10 | 0.08 | 1.76 | −3.01 | −0.28 | 0.27 | 2.09 |
| | −0.78 | 0.16 | 0.39 | 1.19 | −1.13 | 0.09 | 0.42 | 1.67 | −2.53 | −0.27 | 0.48 | 2.99 |
| Ave. | −0.78 | 0.09 | 0.32 | 1.27 | −1.14 | 0.15 | 0.24 | 1.71 | −2.75 | −0.37 | 0.30 | 2.76 |
| Std. | 0.05 | 0.07 | 0.07 | 0.06 | 0.08 | 0.11 | 0.13 | 0.06 | 0.19 | 0.15 | 0.17 | 0.32 |

(Ave. = Average, Std. = Standard deviation)

TABLE 6
Tissue sampling results with the sampling length
lₛ and weight wₛ for SB, LMB, AMB, and HMB stylets.

| | Tissue sample | | | | | Tissue sample | | | |
|---|---|---|---|---|---|---|---|---|---|
| | length lₛ [mm] | | | | | weight wₛ [mg] | | | |
| | SB | LMB | AMB | HMB | | SB | LMB | AMB | HMB |
| | 12.7 | 11.6 | 7.8 | 7.6 | | 7.0 | 8.0 | 4.0 | 4.5 |
| | 12.2 | 11.7 | 10.1 | 6.8 | | 7.0 | 7.0 | 4.0 | 4.5 |
| | 12.6 | 13.9 | 12.4 | 11.2 | | 6.0 | 7.0 | 7.0 | 5.0 |
| | 10.7 | 15.0 | 9.0 | 6.6 | | 8.0 | 7.0 | 6.0 | 4.0 |
| | 12.8 | 13.5 | 8.3 | 10.8 | | 7.0 | 7.0 | 5.0 | 6.0 |
| | 11.9 | 13.6 | 10.5 | 9.7 | | 6.0 | 7.0 | 5.0 | 4.0 |
| | 11.1 | 9.9 | 9.4 | 7.4 | | 7.0 | 6.0 | 5.0 | 6.0 |
| | 12.9 | 14.3 | 10.9 | 10.8 | | 8.0 | 8.0 | 6.0 | 7.0 |
| | 14.1 | 10.8 | 8.8 | 7.2 | | 8.0 | 7.0 | 6.0 | 5.0 |
| | 14.1 | 14.9 | 11.8 | 8.4 | | 7.0 | 8.0 | 7.0 | 6.0 |
| Ave. | 12.5 | 12.9 | 9.9 | 8.6 | Ave. | 7.1 | 7.2 | 5.5 | 5.2 |
| Std. | 1.1 | 1.8 | 1.5 | 1.8 | Std. | 0.7 | 0.6 | 1.1 | 1.0 |

(Ave. = Average, Std. = Standard deviation)

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

We claim:

1. A biopsy device, comprising:
a stylet comprising a cutting tip, wherein said cutting tip comprises at least two bevels and an initial cutting element, wherein at least two of said bevels are radially asymmetric, and wherein said bevels converge to form said initial cutting element.

2. The biopsy device of claim 1, wherein said stylet further comprises a tissue storage groove comprising a tissue groove face, wherein said tissue storage groove is configured for storage of tissue obtained during a biopsy.

3. The biopsy device of claim 1, wherein said initial cutting element is selected from the group consisting of a single cutting point, a horizontal cutting edge, and a vertical cutting edge.

4. The biopsy device of claim 1, wherein said initial cutting element is below or at least partially aligned with the tissue groove face.

5. The biopsy device of claim 1, wherein said at least two bevels comprise at least one primary bevel and at least one balancing bevel.

6. The biopsy device of claim 5, wherein said primary bevel is on the same side of said device as said tissue storage groove.

7. The biopsy device of claim 1, wherein said plurality of balancing bevels generate a force opposite to said primary bevel.

8. The biopsy device of claim 1, wherein said cutting tip comprises one primary bevel and three balancing bevels.

9. The biopsy device of claim 1, wherein said cutting tip comprises one primary bevel and one balancing bevel.

10. The biopsy device of claim 1, wherein said cutting tip comprises one primary bevel and two balancing bevels.

11. The biopsy device of claim 1, wherein said cutting tip comprises two primary bevels and one balancing bevel.

12. The biopsy device of claim 1, wherein said balancing bevels comprise the same or different bevel angle and bevel length.

13. The biopsy device of claims 5 to 11 claim 1, wherein said balancing bevels comprise the same or different bevel angle and bevel length as said primary bevel.

14. The biopsy device of claim 1, wherein a normal surface component of at least one primary bevel is at least partially aligned in the same direction as said a normal surface component of tissue groove face.

15. The biopsy device of claim 1, wherein a normal surface component of at least one balancing bevel is at least partially aligned in the opposite direction as a normal surface component of tissue groove face.

16. The biopsy device of claim 1, wherein said balancing bevels are oriented at plus or minus 90-180° around a center line of the body of said biopsy device relative to said primary bevel.

17-18. (canceled)

19. The biopsy device of claim 1, wherein said biopsy device comprises said stylet, a hollow needle, and a deployment component.

20-21. (canceled)

22. The biopsy device of claim 1, wherein said biopsy device exhibits decreased deflection during deployment relative to a biopsy device lacking said asymmetrical bevels.

23-25. (canceled)

26. A method of obtaining a tissue biopsy sample, comprising: deploying the biopsy device of claim 1.

27. (canceled)

28. A stylet comprising a cutting tip, wherein said cutting tip comprises at least two bevels and an initial cutting element, wherein at least two of said bevels are radially asymmetric, and wherein said bevels converge to form said initial cutting element.

29. (canceled)
